(19) United States
(12) Patent Application Publication
(10) Pub. No.: US 2015/0150525 A1
(43) Pub. Date: Jun. 4, 2015

(54) DYNAMIC NUCLEAR EMISSION AND X-RAY IMAGING DEVICE AND RESPECTIVE IMAGING METHOD

(71) Applicants: Nassir Navab, Munchen (DE); Thomas Wendler, Munchen (DE)

(72) Inventors: Nassir Navab, Munchen (DE); Thomas Wendler, Munchen (DE)

(21) Appl. No.: 14/407,022

(22) PCT Filed: Jun. 11, 2013
(86) PCT No.: PCT/EP2013/062052

§ 371 Date: Dec. 10, 2014

(30) Foreign Application Priority Data
Jun. 11, 2012 (EP) 12171515.5

(51) Int. Cl. A61B 6/00; A61B 6/03

(52) U.S. Cl. CPC A61B 6/4458; A61B 6/037; A61B 6/5205

(57) ABSTRACT

A diagnostic device for computer tomography and nuclear imaging of a body is provided. The device includes at least one movable robot arm, an x-ray source, and a detector for nuclear radiation. Further, a method for the 3D imaging of a body is provided. The method includes irradiating the body by a moving x-ray source, and acquiring nuclear emission data via readings from a moving nuclear detector, supervising the poses of the x-ray source and the nuclear detector, synchronizing the readings from the nuclear detector and the x-ray source with their respective poses, and calculating 3D images by using the acquired information from the x-ray source and from the nuclear detector.

### TECHNICAL FIELD

The present disclosure relates to imaging systems. More particularly, it relates to a device and method for the 3D imaging of a body, in particular a human body, for medical purposes. Even more particularly, it relates to a dynamic nuclear emission and X-ray imaging system and method.

### BACKGROUND OF THE INVENTION

Positron emission tomography (PET) is a nuclear medicine imaging technique that produces a three-dimensional image or picture of functional processes in the body (functional imaging). The system detects pairs of gamma rays emitted indirectly by a positron-emitting radionuclide, which is introduced into the body liked to a biologically active molecule (tracer). Three-dimensional images of tracer concentration within the body are then constructed by computer analysis. The information is typically presented as cross-sectional slices through the patient, but can be freely reformatted or manipulated as required. On occasion, the radionuclide is a simple soluble dissolved ion, such as a radioisotope of gallium (III), which happens to also have chemical properties that allow it to be concentrated in ways of medical interest for disease detection. If the biologically active molecule chosen for PET is 2-fluoro-2-deoxy-D-glucose (FDG), an analogue of glucose, the concentrations of the tracer imaged give tissue metabolic activity, in terms of regional glucose uptake. The use of this tracer to explore the possibility of cancer metastasis (i.e., spreading to other sites) results in the most common type of PET scan in standard medical care.

Another variant of the nuclear imaging is Single-photon emission computed tomography (SPECT), which is also based on radioactive tracers and uses gamma rays, but does not count coincidences like PET, but single particle events on a nuclear radiation detector, making the subsequent analysis more demanding in terms of computing power and complexity of the algorithm involved. It has similarities to conventional nuclear medicine planar imaging using a gamma camera. However, it is able to provide true 3D information.

In modern scanners, PET images are also acquired together with three-dimensional anatomical imaging, which may be accomplished with the aid of a computer tomography (CT) X-ray scan performed on the patient during the same session, in the same machine. X-ray computed tomography means to generate a three-dimensional image of the inside of a body from a large series of two-dimensional X-ray images taken around (typically) a single axis of rotation. CT produces a volume of data that can be manipulated, through a process known as “windowing”, in order to demonstrate various bodily structures based on their ability to block the X-ray beam. Although historically the images generated were in the axial or transverse plane, perpendicular to the long axis of the body, modern scanners allow this volume of data to be reformatted in various planes or even as volumetric (3D) representations of structures.

While the above described combination of techniques in one “hybrid” diagnostic device opens several options for diagnostic purposes, known solutions are, for example, not suitable for the application during surgery. This is mainly because to date, PET and SPECT apparatuses suffer from outer dimensions which typically take up whole rooms and are thus simply not suitable to be used temporarily during a surgery—during which they might be of use several times, but would not be needed in the time in between. Moreover, PET and SPECT devices typically include an examination gantry, in which the patient has to be located during imaging. Of course, this requirement is not compatible with conditions during surgery, as the unconscious patient can not be taken from the operation table and be placed into a nearby imaging device, respectively this would at least require too much time and effort.

In view of the above, there is a need for a “hybrid” imaging technique which avoids disadvantages of the known solutions.

### SUMMARY OF THE INVENTION

The problems mentioned above are at least partly solved by a diagnostic device for computer tomography and nuclear imaging of a body according to claim 1, a use according to claim 13 and a method for the 3D imaging of a body according to claim 14.

In a first aspect, a diagnostic device for computer tomography and nuclear imaging of a body is provided. The device includes at least one movable robot arm, an x-ray source, and a detector for nuclear radiation.

In a second aspect, a method for the 3D imaging of a body is provided. The method includes irradiating the body by a moving x-ray source, providing a radionuclide to the body, and acquiring nuclear emission data via readings from a moving nuclear detector, supervising the poses of the x-ray source and the nuclear detector, synchronizing the readings from the nuclear detector and the x-ray source with their respective poses, and calculating 3D images by using the acquired information from the x-ray source and from the nuclear detector.

In a yet further aspect, a use of a diagnostic device for computer tomography and nuclear imaging of a body is provided.

Further aspects, advantages and features of the present invention are apparent from the dependent claims, the description and the accompanying drawings.

### BRIEF DESCRIPTION OF THE DRAWINGS

A full and enabling disclosure, including the best mode thereof, to one of ordinary skill in the art is set forth more particularly in the remainder of the specification, including reference to the accompanying figures wherein:

FIG. 1 schematically shows a perspective view of a diagnostic device according to embodiments;

FIG. 2 schematically shows a perspective view of a further diagnostic device according to embodiments.

FIG. 3 schematically shows a partial view of a diagnostic device according to embodiments.

### DETAILED DESCRIPTION OF THE INVENTION

Reference will now be made in detail to various embodiments, one or more examples of which are illustrated in each figure. Each example is provided by way of explanation and is not meant as a limitation. For example, features illustrated or described as part of one embodiment can be used on or in conjunction with other embodiments to yield yet further embodiments. It is intended that the present disclosure includes such modifications and variations.

Within the following description of the drawings, the same reference numbers refer to the same components. Generally, only the differences with respect to the individual embodiments are described. When several identical items or parts appear in a figure, not all of the parts have reference numerals in order to simplify the appearance.

The systems and methods described herein are not limited to the specific embodiments described, but rather, components of the systems and/or steps of the methods may be utilized independently and separately from other components and/or steps described herein. Rather, the exemplary embodiment can be implemented and used in connection with many other applications, in particular with other medical diagnostic or treatment methods than the ones exemplarily shown.

As used herein, the term “pose” with reference to an item is defined to be a 6 dimensional (6D) vector including its 3D position with respect to a defined coordinate system and its orientation in 3D. Further, as used herein, “robot” or “robot arm” is defined as a device that can move freely in 6D and is capable of generating several poses of a device mounted to the robot or robot arm. Further, as used herein, “motion” of the interchangeably used terms “a patient”, “patient body”, “body to be examined”, or short “body” is defined as a movement and deformation of anatomy of living beings including, but not constrained to, heart beat and respiration. As used herein, the terms “nuclear detector” and “nuclear radiation detector” and “detector for nuclear radiation” are used interchangeably.

Although specific features of various embodiments of the invention may be shown in some drawings and not in others, this is for convenience only. In accordance with the principles of the invention, any feature of a drawing may be referenced and/or claimed in combination with any feature of any other drawing.

Embodiments of the invention pertain to diagnostic devices for computer tomography and nuclear imaging of a body. They include at least one movable robot arm, to which an x-ray radiation source (x-ray source) and at least one detector for nuclear radiation are mounted or connected. Thereby, the nuclear radiation detector can typically, but not necessarily, detect both gamma radiation emitted by the x-ray source and by a radioactive material, such as a tracer material for SPECT, inside the body to be examined, so that anatomy and functional (here SPECT tracer uptake) components can respectively be imaged. In embodiments, there may also be separate detectors for x-rays and for the detection of the nuclear radiation emitted by the SPECT (or PET) tracer material (radionuclide) inside the patient.

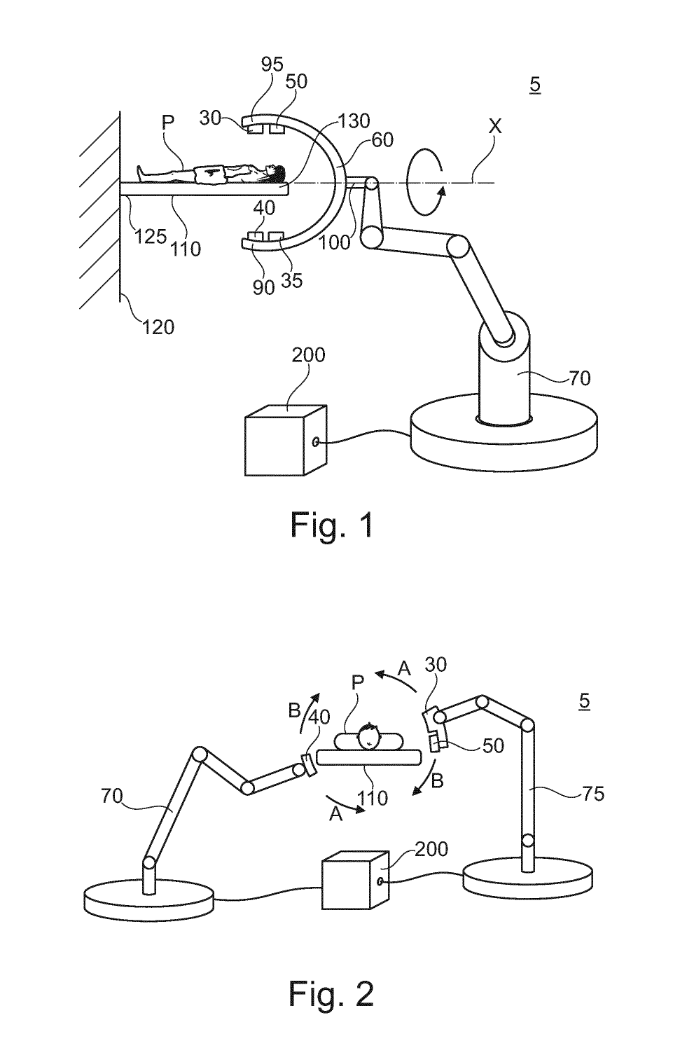

In embodiments, the x-ray source 40 and the nuclear radiation detector 30 are mounted to the at least one robot arm 70, for example to a basically C-shaped, or half circle shaped, member 60 at the end of the arm, such as shown in FIG. 1. The x-ray source 40 may for example be mounted to one end portion 90 of the C as shown, and the nuclear detector 30 is mounted to the other end portion 95 of the “C” member 60, so that they are basically opposite. The C member 60 can be rotatably mounted with its middle portion to the next member 100 of the robot arm 70, so that the C member 60 or half circle can be rotated about its axis of symmetry X by an actuator of the robot arm. The body to be examined P lies on a bed 110 which is typically mounted only at one end to a support structure 120, so that the whole bed has a freely accessible space below (and above) it, except the supporting structure at one end 125.

In embodiments, the robot 70 is typically positioned with respect to the body to be examined P so that the middle portion of the half circle member 60 is located at the freely located end portion 130 of the bed 110, and that the two end portions 90, 95 of the circle member 60 protrude in a direction along the bed 110. If the C member 60 is then rotated about axis X, the source 40 and nuclear detector 30 at the end portions 90, 95 thus move in a circular motion in a plane perpendicular to the longitudinal axis of the bed 110 and the body to be examined P (which are, in their direction, typically roughly similar to axis X).

During such a motion (or a different one, see below with reference to

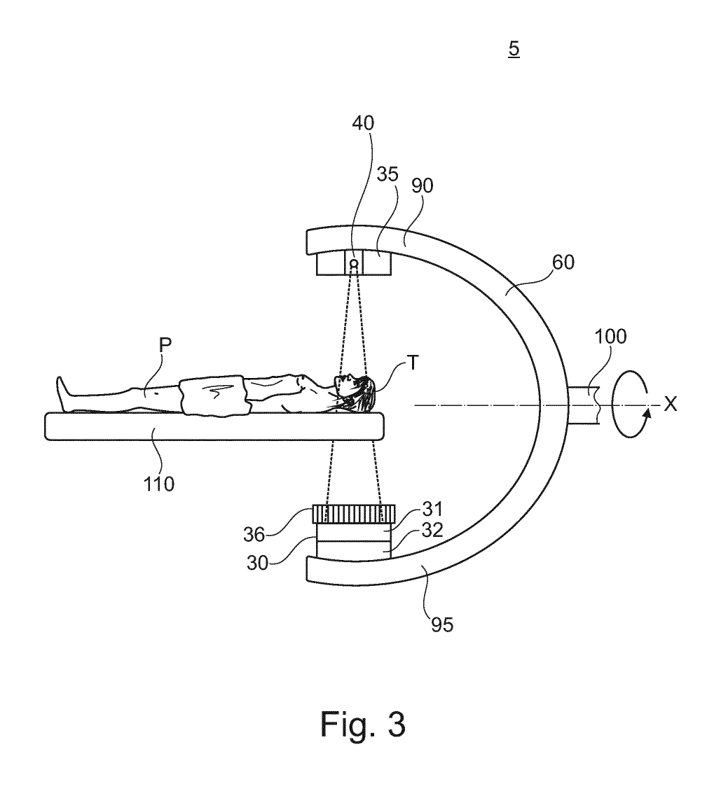

FIG. 2), the x-ray source 40 irradiates the area of interest of the body to be examined, and the nuclear (or eventually extra dedicated x-ray detector 50) detector 30 at the opposite side of the C member 60 detects the transmitted, respectively unabsorbed x-ray radiation through the body. Previously, the body to be examined has been also given a tracer substance (radionuclide) suitable for SPECT detection. The readings from the nuclear detector (and x-ray detector) are continuously monitored by a processing unit 200. If PET is applied, at least two nuclear detectors 30, 35 located at opposite directions from the radionuclide inside the body to be examined are employed (and thus for example at different ends 90, 95 of the C member 60, as shown in FIG. 1 and FIG. 3), for SPECT one detector 30 is sufficient. The data processing unit also monitors the 6D poses of the x-ray source and the nuclear detector and synchronizes the detector readings with the respective pose information, wherein this information is typically stored inside a memory of the unit. The poses can for example be derived from the control electronics of the robot arm, of which position detectors are a typical component. The control electronics is typically at least partly integrated into the data processing unit of the diagnostic device.

The above process of irradiation and acquiring detector readings is continued for a certain time period, which may be predefined by an operator, but which can in embodiments also be controlled by the processing unit, e.g. depending on the quality of the acquired data. I.e., depending on the type and amount of radionuclide tracer, and its concentration in, for example, a tumor in the body to be examined, sufficient data for the required image quality may be obtained after an amount of time which is determined by the processing unit. This time may for example be smaller than average, when the radionuclide is strongly concentrated in a small volume, e.g., a tumor.

Methods for the 3D imaging of a body according to embodiments thus typically include irradiating the body by a moving x-ray source, providing a radionuclide to the body, acquiring nuclear emission data via readings from a moving nuclear detector, supervising the poses of the x-ray source and the nuclear detector, and synchronizing the readings from the nuclear detector and the x-ray source with their respective poses. Via algorithms and methods known to the skilled person, 3D images from the area(s) of interest can be calculated from the data acquired as described above.

Thereby, the simultaneous acquisition of data from the nuclear detector monitoring the gamma rays stemming from decay of the radionuclide, and from x-rays transmitted through the body P to be examined, allows for the simultaneous monitoring of anatomy (via the x-ray computer tomography) and of special interest areas like tumor tissue via the monitoring of the radionuclide concentrated therein. As the data is acquired by sensors and sources mounted to a quickly movable robot arm, the device may be used intermittently during surgery, for example.

Other uses include the use as a positioning aid in radiation therapy. The anatomy and cancer tissue to be cured may be quickly monitored by the devices according to embodiments. Also, surgical instruments may be positioned using the image information by a device according to embodiments, whereby a surgical instrument may in embodiments also be mounted to a further robot arm also operably connected to the processing unit of the diagnostic device. Also, embodiments can be used as a positioning aid in interventional radiology or nuclear medicine, or may deliver image data used as guidance for a physician during intervention or surgery.

In embodiments, the diagnostic device includes two independent robot arms 70, 75, whereby the x-ray source 40 and the nuclear radiation detector 30 are mounted to one arm each as shown in FIG. 2. The movement of the arms is synchronized by the data processing unit 200 or control unit, such that the detector 30 and the x-ray source 40 are moved in circular motions around the body to be monitored. This is schematically shown in FIG. 2 as A and B. The diagnostic device 5 includes (at least) two robot arms 70, 75, one having a nuclear detector 30 (and in embodiments also a dedicated x-ray detector 50), the other having the x-ray source 40. The arms are controlled by the data processing unit 200 and are controlled to carry out circular motions around the patient (directions A and B, respectively). In this configuration, due to mechanical constraints the arms can only cover for example about 180 degrees around the body to be examined P, hence for data acquisition about a period of time, the movements in directions A and B are carried out alternatingly for several times.

In embodiments, the motion of the robot arms is not circular. Accordingly the image generation follows the principles of freehand SPECT for non-symmetric, limited-angle 3D nuclear image reconstruction.

In embodiments, the data processing unit 200 is operable to load previously acquired computer tomography and/or nuclear emission computed tomography images, e.g., from another imaging device. These images can then be updated based on acquired nuclear detector images readings, the pose of the x-ray source and the pose of the nuclear detector. They may also be shown on a display device such as an LCD monitor (not shown).

In embodiments, the diagnostic system has more than one robot arm, and the x-ray source and the nuclear detector (in embodiments combined with the x-ray detector) are mounted to one robot arm each; or are mounted in pairs, i.e., two detectors/sources mounted to one robot arm each.

In embodiments where PET is employed, i.e. where coincident radiation is to be counted in opposite directions from the body P, the nuclear detector comprises at least two separate detector units 30, 35 to detect coincident nuclear emission readings. In general, the nuclear emission computed tomography may be based on at least one of, or a combination of, SPECT, PET, and Compton-Camera image.

In embodiments, the nuclear detector 30, 35 may be configured to detect PET decay events (beta plus decay), and/or SPECT (single photon emissions), and/or to detect Bremsstrahlung of beta minus decay, and/or the x-rays transmitted through body P from the x-ray source. These X-rays may in embodiments be in the same range of emission energy like the single photon emission from the SPECT radionuclide. For example, the X-rays from the source can be at 120 keV, and the gamma particles from the single photon emission may be 140 keV (from radionuclide technetium 99 m). The same nuclear detector 30, 35 may thus in embodiments be used for these two types. Also in case of PET, the same detector 30, 35 may be used for also detecting x-rays. If the detector is for example optimized for PET, the sensitivity for the X-ray images will be lower, but still suffice. Also, if the nuclear detector is optimized for X-rays, it may in embodiments also be used for PET detection with a lower sensitivity.

In embodiments, the different radiation types above (from X-ray, from SPECT, from PET, etc.) may be discriminated by the amplitude of the respective signal, which is commonly proportional to the particle/radiation energy. Such energy windowing is a standard method in X-ray and nuclear imaging.

Hence, in embodiments one nuclear radiation detector 30, 35 will generally suffice to detect the various radiation types according to embodiments. However, for optimizing efficiency and thus to keep reading times lower, distinct detectors 30, 35, 50 may be employed for the distinct radiation types. A combined detector 30, 35 for x-ray and PET is shown schematically in FIG. 3. The detector has a zone 31 optimized for x-ray, e.g. 3 mm thick, and a zone 32 optimized for PET decay, e.g. 5 mm thick. With such a type of detector, sensitivity in both images may be maximized. In FIG. 3 a target structure T is shown, which has a high concentration of the PET radionuclide and thus emits radiation to be sensed by detector part 32 of 30, while the x-ray emitted by source 40 is detected by detector part 31 of detector 30.

Optionally, a collimator 36 may be placed in the radiation path in front of the nuclear detector 30, 35, as shown exemplarily in FIG. 3. The collimator further enhances the spatial resolution of the device. It may be releasably connected to the nuclear detector 30, 35, for example.

In embodiments, the data processing unit 200 is further operable to use x-ray computer tomography data during reconstruction of the nuclear emission computed tomography for attenuation correction and/or the compensation of patient motions. That is, the monitored and calculated attenuation of x-rays by the various body tissues is used in the more complex calculation of the nuclear emission computed tomography.

Typically, in the diagnostic system/device according to embodiments, the data processing unit is operable to acquire x-ray computer tomography data continuously by the x-ray detector and/or nuclear detector during acquisition of nuclear emission readings.

Further, in embodiments, the data processing unit is operable to use x-ray computer tomography images to detect patient motion and trigger a new computer tomography acquisition during a nuclear emission acquisition.

In embodiments, the data processing unit is further operable to determine the quality of x-ray computer tomography and/or nuclear emission computed tomography images during the acquisition. Then, based on the determined quality data such as a calculated quality parameter, the position of the at least one robot arm may be controlled for improving the quality of computer tomography and/or nuclear emission computed tomography images.

In embodiments, there are Compton scattering detectors operably connected to the data processing unit, in order to detect nuclear emission readings coincident with the nuclear detector.

In embodiments, the data processing unit is further operable to acquire computer tomography images depending on the condition that motion of the body is detected. In that manner, the already acquired images can for example be adapted to the new position of the body.

In embodiments, the diagnostic device further includes a collimator. It can be located rigidly or releasably mounted adjacent to the nuclear detector, for example in order to collimate radiation for SPECT readings, in order to improve accuracy. The collimator may also be mounted to a separately controlled further robot arm.

In embodiments, the diagnostic device includes a dedicated further robot arm for positioning a surgical tool in regard to the anatomical region identified from previously acquired 3D images. Thereby, positioning of the tool may be improved and the surgeon effectively assisted.

In embodiments, the diagnostic device further includes a further dedicated robot arm for positioning an additional imaging device. This may be an ultrasound or optical imaging device, for gathering additional information on an anatomical region on which 3D images are acquired by x-ray computer tomography and nuclear emission computed tomography.

In embodiments, a diagnostic device for computer tomography and nuclear imaging of a body includes at least one movable robot arm, an x-ray source, and a detector for nuclear radiation.

In embodiments, a nuclear radiation detector can detect both gamma radiation emitted by the x-ray source and by a radioactive material inside the body.

In embodiments, a diagnostic device further includes an x-ray detector to detect the nuclear radiation emitted by the x-ray source.

In embodiments, a diagnostic device has an x-ray source and nuclear radiation detector mounted to at least one robot arm.

In embodiments, a diagnostic device further has a second robot arm, wherein at least one of the x-ray detector, the x-ray source, and the nuclear radiation detector are mounted to the second robot arm.

In embodiments, a diagnostic device includes a data processing unit, operable to monitor the pose of the x-ray detector, the x-ray source, and the nuclear radiation detector, preferably in a common coordinate system, and further operable to acquire readings from the nuclear radiation detector and to synchronize the readings from the detector and its pose and the pose of the x-ray source.

In embodiments, a diagnostic device has a data processing unit further operable to compute 3D X-ray computed tomography images from the nuclear detector readings, its pose and the pose of the x-ray source.

In embodiments, a diagnostic device has a data processing unit further operable to compute 3D nuclear emission computed tomography images from the nuclear radiation detector readings and the respective poses.

In embodiments, a diagnostic device has a data processing unit further operable to load previously acquired computer tomography and/or nuclear emission computed tomography images; and to update the images based on acquired nuclear detector images readings and the pose of the x-ray source and the pose of the nuclear detector.

In embodiments, a diagnostic device has more than one arm, and the x-ray source, the x-ray detector and the nuclear detector are held in one of the following fashions: a) separately, mounted to one robot arm each; or b) in pairs, two detectors/sources mounted to one robot arm each.

In embodiments, a diagnostic device has a nuclear detector including at least two separate detector units to detect coincident nuclear emission readings.

In embodiments of a diagnostic device, the nuclear emission computed tomography is based on one of the following: SPECT, PET, and Compton-Camera image.

In embodiments, a diagnostic device has a data processing unit operable to use x-ray computer tomography data during reconstruction of the nuclear emission computed tomography for attenuation correction and/or the compensation of motion of the body to be examined

In embodiments, a diagnostic device includes a data processing unit operable to acquire x-ray computer tomography data continuously by the x-ray detector and/or nuclear detector during acquisition of nuclear emission readings.

In embodiments, a diagnostic device has a data processing unit operable to use x-ray computer tomography images to detect patient motion and trigger a new computer tomography acquisition during nuclear emission acquisition.

In embodiments, a diagnostic device has a data processing unit operable to determine the quality of x-ray computer tomography and/or nuclear emission computed tomography images during the acquisition, and to control the position of the at least one robot arm for improving the quality of computer tomography and/or nuclear emission computed tomography images.

In embodiments, a diagnostic device further includes Compton scattering detectors, which are operably connected to the data processing unit to detect nuclear emission readings coincident with the nuclear detector.

In embodiments, a diagnostic device has a data processing unit operable to acquire computer tomography images only under the condition that motion is detected.

In embodiments, a diagnostic device further comprises a collimator, which may be located rigidly or releasably mounted adjacent to the nuclear detector, or mounted to a separately controlled robot arm.

In embodiments, a diagnostic device further comprises a further robot arm for positioning a surgical tool in regard to the anatomical region identified from previously acquired 3D images.

In embodiments, a diagnostic device further comprises a further robot arm for positioning an additional imaging device, preferably an ultrasound or optical imaging device, for gathering additional information on an anatomical region on which 3D images are acquired by x-ray computer tomography and nuclear emission computed tomography.

In embodiments, a computer program product comprises computer program code that, when executed on a computer, will control a diagnostic device according to embodiments described herein.

In embodiments, a diagnostic device according to embodiments is used as a positioning aid in radiation therapy and/or as a positioning aid in surgery, and/or as a positioning aid in interventional radiology/nuclear medicine and/or as image-guidance in surgery.

In embodiments, a method for the 3D imaging of a body includes irradiating the body by a moving x-ray source, providing a radionuclide to the body, acquiring nuclear emission data via readings from a moving nuclear detector, supervising the poses of the x-ray source and the nuclear detector, synchronizing the readings from the nuclear detector and the x-ray source with their respective poses, calculating 3D images by using the acquired information from the x-ray source and from the nuclear detector.

In embodiments of a method for the 3D imaging of a body, an x-ray source and a nuclear detector are mounted to at least one robot arm and are moved in a circular motion each around the body during irradiation and reading.

In embodiments of a method for the 3D imaging of a body, the motion of the robot arms is not circular.

The methods described according to embodiments described herein may also be embodied in a computer program product, which includes computer program code that, when executed on a data processing unit, will control a diagnostic device according to embodiments described herein.

This written description uses examples to disclose the invention, including the best mode, and also to enable any person skilled in the art to practice the invention, including making and using any devices or systems and performing any incorporated methods. While various specific embodiments have been disclosed in the foregoing, those skilled in the art will recognize that the spirit and scope of the claims allows for equally effective modifications. Especially, mutually non-exclusive features of the embodiments described above may be combined with each other. The patentable scope of the invention is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal language of the claims.

1. Diagnostic device for computer tomography and nuclear imaging of a body, comprising:
at least one movable robot arm,
an x-ray source, and
a nuclear radiation detector;
wherein either
a) the nuclear radiation detector is adapted to detect gamma radiation emitted by a SPECT/PET radionuclide tracer material inside a body to be examined and gamma radiation emitted by the x-ray source, so that it is also an x-ray detector; or
b) the nuclear radiation detector is adapted to detect gamma radiation emitted by the SPECT/PET radionuclide tracer material, and the diagnostic device further comprises a dedicated x-ray detector for detecting gamma radiation emitted by the x-ray source.

2. Diagnostic device according to claim 1, wherein x-ray source and nuclear radiation detector are mounted to the at least one robot arm, and wherein optionally the robot has more than one arm, and the x-ray source, the nuclear radiation detector, and optionally the dedicated x-ray detector are held in one of the following fashions:
separately, mounted to one robot arm each;
in pairs, two detectors/sources mounted to one robot arm each.

3. Diagnostic device according to claim 1, further comprising:
a data processing unit, operable to monitor the pose of the x-ray source and the nuclear radiation detector, preferably in a common coordinate system, and further operable to acquire readings from the nuclear radiation detector and the optionally dedicated x-ray detector and to synchronize the readings from both the detectors and their poses and the pose of the x-ray source, wherein the x-ray detector and the nuclear radiation detector may be the same.

4. Diagnostic device according to claim 3, wherein the data processing unit is further operable to compute 3D X-ray computed tomography images from the x-ray detector readings, its pose and the pose of the x-ray source, and/or wherein the data processing unit is further operable to compute 3D nuclear emission computed tomography images from the nuclear radiation detector readings and the respective poses.

5. Diagnostic device according to claim 3, wherein the data processing unit is further operable to load previously acquired computer tomography and/or nuclear emission computed tomography images;
and to update the images based on acquired nuclear radiation detector readings and x-ray detector readings, their poses and the pose of the x-ray source.

6. Diagnostic device according to claim 1, wherein the nuclear radiation detector comprises at least two separate detector units to detect coincident nuclear emission readings, and wherein the nuclear emission computed tomography is based on one of the following: SPECT, PET, and Compton-Camera image.

7. Diagnostic device according to claim 3, wherein the data processing unit is operable to use x-ray computer tomography data during reconstruction of the nuclear emission computed tomography for attenuation correction and/or the compensation of motion of the body to be examined, and wherein the data processing unit is optionally operable to use x-ray computer tomography images to detect motion of the body to be examined and trigger a new computer tomography acquisition during nuclear emission acquisition, wherein the data processing unit is operable to acquire computer tomography images only under the condition that motion is detected.

8. Diagnostic device according to claim 3, wherein the data processing unit is operable to determine the quality of x-ray computer tomography and/or nuclear emission computed tomography images during the acquisition, and to control the position of the at least one robot arm for improving the quality of computer tomography and/or nuclear emission computed tomography images.

9. Diagnostic device according to claim 1, further comprising Compton scattering detectors, which are operably connected to the data processing unit to detect nuclear radiation detector readings and x-ray detector readings, the poses of Compton scattering detectors, the poses of the nuclear detector and x-ray detector, and the pose of the x-ray source, coincident with the nuclear detector.

10. Diagnostic device according to claim 1, further comprising a collimator, located:
rigidly or releasably mounted adjacent to the nuclear detector,
mounted to a separately controlled robot arm.

11. Diagnostic device according to claim 1, further comprising a further robot arm for positioning a surgical tool in regard to the anatomical region identified from previously acquired 3D images, and/or for positioning an additional imaging device, preferably an ultrasound or optical imaging device, for gathering additional information on an anatomical region on which 3D images are acquired by x-ray computer tomography and nuclear emission computed tomography.

12. A computer program product, comprising computer program code that, when executed on a computer, will control a diagnostic device according to claim 1.

13. Use of a device according to claim 1:
as a positioning aid in radiation therapy and/or,
as a positioning aid in surgery and/or,
as a positioning aid in interventional radiology/nuclear medicine and/or,
as image-guidance in surgery.

14. Method for the 3D imaging of a body, comprising:
irradiating the body by a moving x-ray source,
providing a radionuclide to the body,
acquiring nuclear emission data via readings from a moving nuclear detector,
supervising the poses of the x-ray source and the nuclear detector,
synchronizing the readings from the nuclear detector and the x-ray source with their respective poses,
calculating 3D images by using the acquired information from the x-ray source and from the nuclear detector.

15. Method for the 3D imaging of a body according to claim 14, wherein the x-ray source and the nuclear detector are mounted to at least one robot arm and are moved each around the body during irradiation and reading.
